(19) United States
(12) Patent Application Publication
(10) Pub. No.: US 2023/0256192 A1
(43) Pub. Date: Aug. 17, 2023

(54) SYSTEMS AND METHODS FOR INDUCING SLEEP OF A SUBJECT

(71) Applicant: KONINKLIJKE PHILIPS N.V., EINDHOVEN (NL)

(72) Inventors: MERT BEREKET, EINDHOVEN (NL); VINCENTIUS PAULUS BUIL, VELDHOVEN (NL)

(21) Appl. No.: 18/009,180

(22) PCT Filed: Jun. 17, 2021
(86) PCT No.: PCT/EP2021/066374

§ 371 Date: Dec. 8, 2022

Related U.S. Application Data
(60) Provisional application No. 63/041,304, filed on Jun. 19, 2020.

(51) Int. Cl. A61M 21/02; A61B 5/00

(52) U.S. Cl. CPC A61M 21/02; A61B 5/486; A61M 2021/0027

(57) ABSTRACT

Systems and methods are proposed for generating a sleep-inducing audio signal for a subject. Such concepts may aid and/or induce sleep through the provision of subject-specific (i.e. personalized) audio that is based on psychological states previously experienced by the user (e.g. in the preceding day or week). In particular, embodiments propose that sounds experienced by a subject when in a target psychological state (e.g. positive, feel-good state) may be captured and used to generate an audio signal that may have improved sleep inducing capabilities/qualities.

### FIELD OF THE INVENTION

The invention relates to human sleep, and more particularly to the field of aiding or inducing sleep of a human subject.

### BACKGROUND OF THE INVENTION

Systems and methods have been proposed for promoting and aiding healthy sleep habits in human subjects. By way of example, systems exist that are configured to provide a sensorial ambient experience and gently coach a user to have healthy sleep habits.

Known approaches to aiding and/or inducing sleep have typically focused on facilitating relaxation through light and sound stimuli and/or through cleaning bedroom air (e.g. for improving a quality of a user's sleeping environment. One such system employs a software application for a mobile computing device, wherein the application invites/prompts a user to go and provides an overview of personalized sleep insights. The software application also controls the mobile computing device and/or a connected device to provide a sleep-inducing ambient environment (e.g. by controlling sounds and/or lighting provided to the user in his/her bedroom environment). For example, devices exist that gently dim lighting and/or adjust the lighting color to a calming red range.

### SUMMARY OF THE INVENTION

The invention is defined by the claims.

According to examples in accordance with an aspect of the invention, there is provided a method for generating a sleep-inducing audio signal for a subject, the method comprising:

monitoring a psychological state of a subject during a predetermined time period to detect if the psychological state of the subject matches a target psychological state;

responsive to detecting that the psychological state of the subject matches a target psychological state, obtaining an audio recording of the surrounding environment of the monitored subject, wherein the audio recording is captured at a detection time during which the psychological state of the subject matches the target psychological state; and

generating an audio signal for the subject based on the audio recording and the one or more predetermined sleep inducing sounds.

Embodiments propose concepts for aiding and/or inducing sleep through the provision of subject-specific (i.e. personalized) audio. Such proposals are based on a realization that a sounds experienced by a subject when in a positive psychological state may be leveraged to aid or induce the subject's sleep. That is, proposed are concepts for helping a user to enter a positive or restful psychological state thought the use of sounds that the user may remember or associate from/with a previously-experienced positive or restful psychological state. For example, an audio recording may be captured from the surrounding environment of the subject when he/she is detected as being in a positive (e.g. happy, feel-good, rested, etc.) mental state, and this audio recording may then be used to generate sound(s) that can be provided to the subject to aid or assist sleep inducement. Such an example may help a user to unwind and/or feel happy when reflecting on his/her positive moments before falling asleep.

For example, unlike conventional approaches, embodiments may facilitate the provision of a listening experience that is tailored (e.g. personalized) to a subject's own positive sound memories. Also, embodiments may generate sleep-inducing sounds that are shaped by environmental, psychological and physiological data.

Proposed embodiments may facilitate the provision of dynamically-changing layers of sounds that are captured from monitoring a user's physiological and environmental data throughout a monitoring time-period (e.g. the daytime). Such sounds may then be delivered when the user is getting ready to sleep. For instance, an embodiment may be configured to create a personalized sound experience at the end of day, wherein the sound experience contains sounds captured during the day when the user was in a positive psychological state (e.g. good mood, or happy mindset).

Embodiments thus propose one or more concepts for an adaptive audio signal generation method which makes use of sounds experienced by a monitored subject when he/she was in a target psychological state of the subject. Such sounds may be automatically captured (i.e. recorded) whenever the subject is identified as being in the target psychological state). Also, the subject may control the capture of such sounds by providing an input signal that indicates he/she is in the target psychological state. That is, embodiments may cater for subjects actively controlling the recording of sounds. Furthermore, embodiments may also use sound recordings from elsewhere to tailor the audio signal (and/or a listening experience provided by the audio signal). For example, sounds from video recordings on a smartphone, sounds from a multimedia document available on the internet, etc. may be employed). Put another way, embodiments may capture ‘feel good’ sounds automatically, and also allow a subject to record and/or their own sounds. Embodiments may therefore facilitate the creation and use of personalized and/or adaptive sleep inducing audio signals.

As a result, systems and methods are proposed for audio-based sleep inducement. Such proposals may be based on obtaining an audio recording of the surrounding environment of a monitored subject, whenever the subject is in a target psychological state (e.g. feel-good mood, relaxed mindset, happy mood). Such obtained sound(s) may then be used to generate an audio signal for the subject, for example by mixing the sound(s) other, known sleep inducing sounds (such as pink noise; ambient music; and nature sounds for example). By providing the generated audio signal to a speaker, am improved sleep inducing environment/experience may be provided to the subject when he/she is trying to get to sleep.

Embodiments may therefore be based on using sound experienced by subject (e.g. human user) when in a positive or restful psychological state. Such embodiments may cater for the fact that human memories can be recalled/prompted by sounds associated with the memories, thus facilitating the prompting or recollection of positive or restful memories/thoughts that may aid or induce sleep. Such recollections/memories/thoughts may also significantly reduce stress (which is often elevated before sleep). Proposed implementations may therefore be useful for reducing stress (e.g. mental rumination) that can be experienced by a subject before sleep.

Regular use of embodiments may also help a subject to experience more positive emotions outside of the sleep context. For instance, repeated, regular use (e.g. daily, or weekly) of proposed embodiments may sensitize a user/subject to their positive memories and facilitate Cognitive Behavior Therapy.

Proposed embodiments may thus provide dynamic and/or adaptive sleep inducing concepts that cater for changing psychological states experienced by a subject.

It is also proposed that existing sleep inducing apparatus may be improved or augmented through the use of a method and/or system according to an embodiment. That is, proposed embodiments may be integrated into (or incorporated with) conventional sleep inducing systems to improve and/or extend functionalities.

Embodiments may therefore be of particular use in relation to sleep inducing apparatus that is designed to facilitate and/or promote relaxation through light and sound stimuli. Exemplary usage applications may, for example, include the use of a generated audio signal to inducing sleep in a subject. Embodiments may thus be of particular use in relation to personal health care and/or sleep assistance.

Some embodiments may further comprise: responsive to detecting that the psychological state of the subject matches a target psychological state, determining a weather condition experienced by the subject at the detection time and identifying a weather sound based on the determined weather condition. Generating an audio signal for the subject may then be further based on the weather sound. Embodiments may therefore leverage weather data to further adapt and/or personalize the audio signal to an experience or memory of the subject. Such embodiments may be based on a proposal that a subject may associate weather conditions with positive memories or good mental states. Use of such weather conditions may thus improve recollection of positive memories or happy feelings, which in turn may improve sleep inducement.

In some embodiments, the monitoring a psychological state of the subject comprises monitoring at least one of: subject location; time; one or more vital signs of the subject; one or more short-range communication links established with a computing device carried by the subject; subject brainwaves (e.g. to provide information about stress arousals, relaxation states, etc.); environmental data (e.g. pollution, noise levels, etc.); user/subject input (e.g. indications of a psychological state, indications of how much the subject likes an experience, indications of sound content rated/ranked by the subject, user/subject feedback on detected sounds or events, etc.); physiological parameters (e.g. skin conductance, respiration rate, oxygen saturation, blood pressure, etc.); detected sounds (e.g. indicators of events, including laughter, spoken word, shouting, etc); scheduled activities (e.g. planned events, planned holidays, scheduled meetings, important dates such as birthdays, anniversaries, etc.) and weather conditions experienced by the subject. Embodiments may therefore leverage various different approaches to monitoring the psychological state of the user, and such approaches may be known and/or widely available (thus reducing cost and/or complexity of implementation). For instance, monitoring of a psychological state of a subject may be achieved by monitoring physiological, environmental, and contextual data of the subject. From such various sources of data, inferences and/or determinations may be made about the psychological state of the subject, thus enabling monitoring of the psychological state. Further, use of different approaches may improve accuracy of monitoring and/or accuracy of psychological state detection/determination.

Generating an audio signal for the subject may be further based on predetermined sound cues for indicating respiration actions. For example, the audio signal may be generated to include sound cues that help guiding paced breathing exercises (for reducing stress and/or relaxing the body and mind of the subject). Breathing exercises may, for instance employ sound cues that mark each inhale-exhale moments, depending on a selected breathing exercise.

In some embodiments, generating an audio signal may comprise mixing the audio recording and the one or more predetermined sleep inducing sounds. For example, sleep inducing sounds may be mixed with sound content (e.g. audio recordings) captured for the monitored time period. The sleep inducing sounds may create the foundation of the track, so that their presence may be more dominant then the audio recording(s). Further, the sleep inducing sounds may be configured be longer (in duration) than other sounds or recordings that are mixed to generate the audio signal (e.g. to provide room to a user to slow down his/her thinking and relax after hearing a personal memory (i.e. audio recording). In some examples, mixing may comprise defining a tempo of the audio signal based on at least one of: a preference of the subject; and detected value of a physiological parameter of the subject. In this way, the audio signal may be tailored to preferences and/or user needs, so as to improve sleep inducement. For instance, a daily average heart rate variability may be used to define the tempo of the audio signal in direct proportion between the range of 50-60 beats per minute. This rhythmic element may be created by binaural beats, and the tempo and the audio recording(s) may then help to reduce the subject's heart rate and/or relax breathing for stress reduction.

The process of monitoring may be controlled responsive to an indication or instruction provided by the subject. In this way, the subject may actively control one or more aspects of audio signal generation, thus catering for user preferences and/or requirements.

In an embodiment, obtaining an audio recording of the surrounding environment of the monitored subject may comprise capturing sound with a microphone carried by the subject. Embodiments may therefore employ existing equipment that is widely available, thus reducing cost and/or complexity of implementation. For instance, embodiments may leverage the realization that most subjects typically carry a portable computing device (e.g. mobile phone or tablet) that is capable of capturing sound from a surrounding environment (e.g. via a built-in microphone). Embodiments may therefore make use of equipment/apparatus already provisioned by the subject.

By way of example, the one or more predetermined sleep inducing sounds may comprise at least one of: pink noise; binaural beats; ocean sounds; ambient music; and nature sounds. Embodiments may therefore employ various sleep inducing sounds that are already well-known and/or available. Such sounds may be used to form the foundation of an audio signal which can then be further tailored and/or improved for the subject through the addition of one or more audio recordings captured from the surrounding environment of the subject when he/she was in the target psychological state (e.g. good mood or happy mindset).

Some embodiments may further include the step of communicating the generated audio signal to a sleep inducing apparatus. Embodiments may therefore be adapted to provide a generated audio signal as an output signal for use by another system (e.g. conventional sleep inducing apparatus). In this way, embodiments may provide improved and/or additional functionality to existing apparatus.

According to examples in accordance with yet another aspect of the invention, there is provided a computer program product comprising computer program code means which, when executed on a computing device having a processing system, cause the processing system to perform all of the steps of the method described above.

According to another aspect of the invention, there is provided a system for generating a sleep-inducing audio signal for a subject, the system comprising:

a monitoring component configured to monitor a psychological state of a subject during a predetermined time period to detect if the psychological state of the subject matches a target psychological state;

an interface configured, responsive to detecting that the psychological state of the subject matches a target psychological state, to obtain an audio recording of the surrounding environment of the monitored subject, wherein the audio recording is captured at a detection time during which the psychological state of the subject matches the target psychological state; and

a audio processor configured to generate an audio signal for the subject based on the audio recording and one or more predetermined sleep inducing sounds.

In some embodiments, the audio processor may comprise an audio mixer configured to mix the audio recording and the one or more predetermined sleep inducing sound.

Some embodiments of the system may comprise an output interface configured to communicating the generated audio signal to a sleep inducing apparatus.

The system may be further configured to generate a control instruction for a sleep equipment based on the generated audio signal. In this way, sleep equipment may be controlled according to instructions generated by embodiments (e.g. controlled to play one or more sounds provided in/by a generated audio signal). Dynamic and/or automated control concepts may therefore be realized by proposed embodiments.

Further, proposed concepts may provide a sleep aid (or sleep support apparatus) comprising a system according to a proposed embodiment. For instance, according to yet another aspect of the invention, there is provided a sleep inducing apparatus comprising a system for generating a sleep-inducing audio signal for a subject according to a proposed embodiment.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a flow diagram of a method according to a proposed embodiment;

FIG. 2 depicts a modification to the method of FIG. 1;

FIG. 3 is a simplified block diagram of a system according to an embodiment; and

FIG. 4 is a simplified block diagram of a computer within which one or more parts of an embodiment may be employed

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for generating a sleep-inducing audio signal for a subject. Such concepts may aid and/or induce sleep through the provision of subject-specific (i.e. personalized) audio that is based on psychological states previously experienced by the user (e.g. in the preceding day or week).

In particular, embodiments propose that sounds experienced by a subject when in a target psychological state (e.g. positive, feel-good state) may be captured and used to generate an audio signal that may have improved sleep inducing capabilities/qualities. For instance, embodiments may be configured to help a subject to enter a positive or restful psychological state thought the use of sounds that the subject may remember or associate from/with a previously-experienced positive or restful psychological state.

Whereas conventional approaches may employ stored template sleep inducing sounds, proposed concepts are based on a realization that a personalized sound experience may be provided to a subject, wherein the sound experience contains sounds heard/experienced by the subject when he/she was in a positive psychological state (e.g. good mood, or happy mindset). Proposed concepts may also use additional features experienced by the subject when in a positive psychological state (e.g. weather conditions) to further adapt an audio signal for inducing sleep in the subject.

Based on such proposals, embodiments may provide a concept of generating a sleep-inducing audio signal for a subject based on an audio recording of the surrounding environment of the monitored subject, the audio recording being captured when the psychological state of the subject matches a target psychological state (e.g. happy state, relaxed state, feel-good mood, etc.).

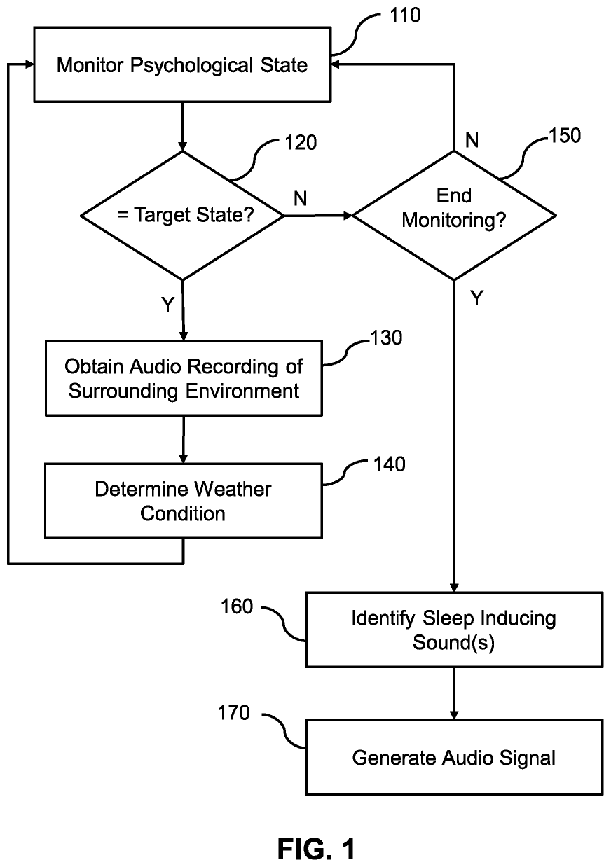

Referring now to FIG. 1, there is depicted a flow diagram of a method for verifying for generating a sleep-inducing audio signal for a subject according to exemplary embodiment. In this example the subject is an adult human (e.g. patient) who typically wears a smartwatch throughout the entirety of his/her normal day. That is, while undertaking normal activities of daily living, the subject has a smartwatch on his/her person at all times. The smartwatch is loaded with a software application (i.e. app′) that may be configured to implement part (or all) of the method of this exemplary embodiment. The smartwatch also comprises a microphone for recording sound (i.e. capturing audio recordings of the surrounding environment), along with other apparatus/components that may be employed for monitoring the subject (e.g. accelerometer(s), heart rate sensor, geographic location sensors, temperature sensor, vital sign sensor, etc.).

The begins with the step 110 of monitoring a psychological state of a subject during a predetermined time period to detect if the psychological state of the subject matches a target psychological state (such as a rested or relaxed state). In this example, the step 110 of monitoring a psychological state of the subject comprises monitoring one or more the following: subject location; one or more vital signs of the subject; one or more short-range communication links established with a computing device carried by the subject; and weather conditions experienced by the subject.

In step 120 it is determined whether the monitoring has detected that that the psychological state of the subject matches the target psychological state. If it is determined in step 120 that the psychological state of the subject matches the target psychological state, the method proceeds to steps 130 and 140.

Step 130 comprises obtaining an audio recording of the surrounding environment of the monitored subject. Here, the audio recording is captured at a detection time during which the psychological state of the subject matches the target psychological state. That is. Specifically, in this example, obtaining an audio recording of the surrounding environment of the monitored subject comprises capturing sound with a microphone carried by the subject, namely the microphone of the subject's smartwatch.

Step 140 comprises determining a weather condition experienced by the subject at the detection time and identifying a weather sound based on the determined weather condition. For instance, in this exemplary embodiment, the application uses an internet connection (e.g. provided via the subject's cellular phone/device) to retrieve a description of the current weather conditions (i.e. the local weather conditions prevailing at the detection time) from an internet-based meteorological service.

After completing steps 130 and 140, the method returns to step 110 and the monitoring of the subject psychological state repeats.

If it is determined in step 120 that the psychological state of the subject does not match the target psychological state, the method proceeds to step 150, wherein it is determined whether or not the predetermined time period has fully elapsed (i.e. the monitoring period has ended). If it is determined in step 150 that the predetermined time period has not fully elapsed (i.e. the predetermined time period has not ended), the method returns to step 110 and the monitoring of the subject psychological state repeats. Conversely, if it is determined in step 150 that the predetermined time period has fully elapsed (i.e. the predetermined time period has ended), the methods can then proceed to steps 160 and 170. Here it is noted the method may delay execution of steps 160 and/or 170 until prompted or instructed. For example, the method may proceed to execute step 160 responsive to the subject providing an indication (e.g. input signal) that sleep assistance is requested/desired.

Step 160 comprises identifying one or more predetermined sleep inducing sounds. Such sounds may already be pre-selected according to subject-preferences, and thus automatically identified and retrieved from a data store/library. In other examples. the step of identifying may comprise prompting the user to choose/select sleep inducing sounds from an available set of sleep inducing sounds. In this example, the one or more predetermined sleep inducing sounds comprise at least one of: pink noise; binaural beats; ocean sounds; ambient music; and nature sounds. Such sleep inducing sounds are example of known/conventional sounds that may assist in inducing sleep (when employed appropriately). The identified sleep inducing sounds may be employed to form the foundation (e.g. base soundtrack) of an audio signal.

Step 170 comprises generating an audio signal for the subject based on the audio recording and the one or more predetermined sleep inducing sounds. Specifically, in this exemplary embodiment, the step of generating an audio signal comprises mixing: the audio recording(s) obtained from step 130; the weather sound(s) from step 140; and sleep inducing sound(s).

For example, where the sleep inducing sounds are employed to form the foundation (e.g. base soundtrack) of an audio signal, the audio recording(s) and the weather sound(s) are then added to (e.g. audio mixed with) the sleep inducing sound(s) to create a final audio signal that is tailored to the subject. Put another way, a base/backing audio track of the sleep inducing sound(s) is adapted and/or improved for the subject through the addition of the audio recording(s) captured from the surrounding environment of the subject when he/she was in the target psychological state (e.g. good mood or happy mindset). This is then further adapted and/or improved through the addition of weather sound(s) experienced by the subject when he/she was in the target psychological state (e.g. good mood or happy mindset).

It is to be understood that the above-described embodiment of FIG. 1 is purely exemplary. Such an embodiment may therefore be adapted and/or modified according to requirements and/or desired application. Also, the method of FIG. 1 is only illustrative and the ordering of one or more of the steps may be modified in alternative embodiments. For example, steps 130 and 140 may be undertaken in parallel. In other embodiments, step 160 may be omitted and a fixed, pre-selected set of sleep inducing sounds employed, for example.

By way of example, it is proposed that sleep inducing apparatus may be controlled according to an audio signal generated according to an embodiment. For example, some embodiments may further comprising communicating the generated audio signal to a sleep inducing apparatus.

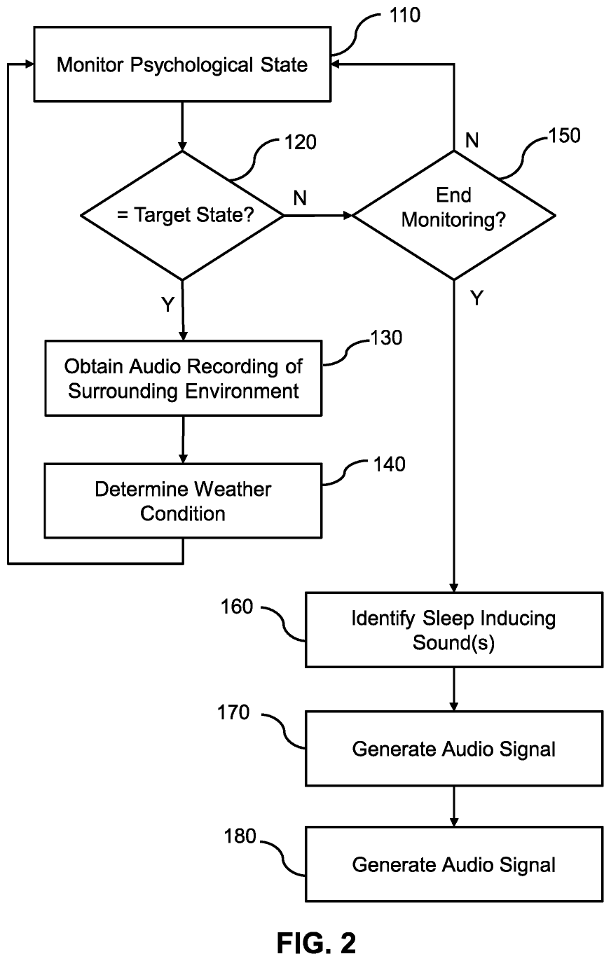

For instance, referring to FIG. 2, there is depicted a flow diagram of a method according to another embodiment, wherein the method is a modified version of the method of FIG. 1. Specifically, the embodiment of FIG. 2 comprises all of the steps of the method of FIG. 1, but further comprises the step 170 of communicating the generated audio signal to a sleep inducing apparatus.

In a further example, it is proposed that the mixing in step 170 may comprise defining a tempo of the audio signal based on at least one of: a preference of the subject; and detected value of a physiological parameter of the subject. In this way, the audio signal may be tailored to a user preferences and/or needs. For instance, a daily average heart rate variability may be used to define the tempo of the audio signal. A rhythmic element may be created by binaural beats for example, and the tempo combined with the audio recording(s) may then improve sleep inducement (e.g. by helping to reduce the subject's heart rate and/or relax breathing for stress reduction).

One or more other devices may be employed instead of a smartwatch. For instance, embodiments may leverage components and/or capabilities of a smartphone (such a integrated processor, data storage, communication link, microphone, speaker, accelerometer, pulse rate monitor, etc.). Other embodiments may employ ‘smart glasses’ and/or other wearable or portable computing devices, as well as stationary devices such as in-home, outdoor or public (security) cameras (e.g. facial expression recognition to detect smiling, or camera based breathing and heart rate detection), microphones or other relevant remote sensing technologies.

According to yet another example, it is proposed that generating the audio signal for the subject may be further based on predetermined sound cues for indicating respiration actions. For instance, the audio signal may be generated to include sound cues for guiding paced breathing exercises. Such breathing exercises may employ sound cues that mark inhalation-exhalation moments (e.g. depending on a selected breathing exercise).

It is also proposed that the monitoring of psychological state may be controlled responsive to an indication or instruction provided by the subject. For instance, the subject may actively control (e.g. pause, start, end and/or correct) the detection of the target psychological state. Alternatively, or additionally, the target state may be defined and/or modified based on an indication or instruction provided by subjects. Supervised learning concepts may therefore be employed to improved accuracy of detecting the target psychological state.

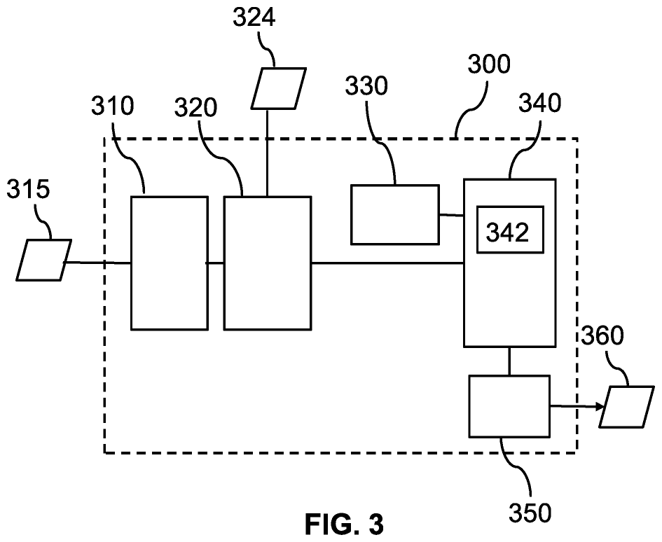

Referring now to FIG. 3, there is depicted a simplified block diagram of a system according to an embodiment. The system is for generating a sleep-inducing audio signal for a human subject. The system 300 comprises a monitoring component 310 configured to monitor a psychological state of a subject during a predetermined time period to detect if the psychological state of the subject matches a target psychological state. In this example, the monitoring component 310 is configured to receive one or more signals from a vital signs sensor 315 worn/carried by the subject.

The system 300 also comprises an interface 320 that is configured, responsive to detecting that the psychological state of the subject matches a target psychological state, to obtain an audio recording 324 of the surrounding environment of the monitored subject. Here, the interface 320 is configured to retrieve an audio recording 324 is captured at a detection time during which the psychological state of the subject matches the target psychological state.

A processing unit 330 of the system 300 is configured to identify one or more predetermined sleep inducing sounds based on an input provided by the subject. That is, based on an indication of preference provided by the subject, the processing unit 330 is adapted to retrieve one or more sounds from a set of available sleep inducing sounds. Such a set of sleep inducing sounds may be available locally (i.e. stored in a data storage unit of the processing unit of the system 300) and/or available in a remote store/library (e.g. stored in a remote data store accessible via the Internet or in a cloud-based computing system). In other embodiments, the sounds may already be pre-defined, and simply made available by the processing unit.

The system 300 also comprises an audio processor 340 that is configured to generate an audio signal 360 for the subject based on the audio recording and the one or more predetermined sleep inducing sounds. In this example of FIG. 3, the audio processor 340 comprises an audio mixer 342 that is configured to mix the audio recording and the one or more predetermined sleep inducing sound so as to generate the audio signal 360.

The system 300 further comprises an output interface 350 that is configured to output the audio signal 360 (e.g. to provide the audio signal 360) to a sleep inducing apparatus.

From the above-described examples, it will be understood that one or more concepts are proposed wherein a subject's listening experience can be tailored to his/her own positive sound memories. This may be done by shaping sleep-inducing sound by environmental and/or physiologic data captured for the subject in a preceding timeframe (e.g. preceding daytime).

By way of further example, layers of sounds may be processed/modified based on results of monitoring the subject's physiological and environmental data throughout the daytime. The processed sounds may then be delivered (i.e. output via a speaker) to the subject when getting ready to sleep.

Embodiments may aim to capture sounds from daily ‘feel-good’ moments and then re-deliver the sounds to the subject in a sleep inducing. In this way, a personalized sleep-inducing sound experience may be provided that helps the subject fall asleep at ease. For instance, embodiments may generate a sound signal that provides an aural experience that steers thoughts to a positive reflection state, and this may be done by curating sound recordings of the subject's happy moments during a preceding time periods (such the daytime or preceding days/weeks, for example).

An aural experience provided by a sound signal generated by an embodiment may therefore be a mixture of sleep inducing sounds and sound recordings of from the subject's daily life. Furthermore, the sleep inducing sounds and sound recordings from positive moments may also be mixed with sound cues that help to guide paced breathing exercises. In this way, a generated sound signal may facilitate a breathing exercise that helps to reduce stress and relax the body and/or mind of the subject.

In some embodiments, the audio signal may be generated so that, for a first part of the listening experience, audio recordings from occurrences of positive psychological states are blended with sleep-inducing sounds. In a second part of the listening experience, the sleep-inducing sounds may remain, and sound cues for breathing exercises may be added to mark breathing moments.

To detect psychological state (and thus determine if a subject is in a target psychological state), it is proposed that various forms of data and/or parameters may be monitored and assessed. Purely by way of example, data regarding subject location, heart rate, weather conditions and may be leveraged. Such data/information may be used to automate the capture (i.e. recording) of audio recordings from ‘feel good’ (e.g. happy, relaxed, blissful, joyful or content) moments. Capturing such ‘feel good’ moments as sounds may employ various components that are configured to detect data and to process the captured data. Such components may, for example, by provided by an activity tracker and/or smartphone

Purely to provide further examples and explanations, various aspects or components of potential embodiments will now be described in the following titled sections. Such sections simply detail exemplary implementations and/or modifications by way of demonstrating various options for employing the proposed concept(s).

Detecting Target Psychological Stats (e.g. ‘Feel Good’ Moments) with

Algorithms

When the subject first uses an embodiment a user interface may be provided for accepts inputs and displaying outputs. This may enable the subject to configure various aspect and/or options for implementation.

For example, an embodiment may seek permission to tracks the subject's location. This way, such an embodiment may detects the subject's frequent locations as well as notable/unexpected changes in movement/travel patterns, such as a nature trip, museums, day/night time recreational outings. Also, certain locations may have negative associations such as hospitals, the cemetery, etc. thus enabling inference of a potentially negative psychological state.

Such information about locations/points of interest may be defined within location Application Programming Interface (API) service that can be used through third-party applications. Moreover, known feedback-based machine learning algorithms may be adopted so that embodiments can learn from the subject's behavior.

Embodiments may also allow a subject to save favorite locations and add exceptions on-demand to respect privacy, e.g. frequently visited location asked by the system: “Do you want to save it as a favorite location?” Frequently visited location detected during working hours and asked by the system: “Do you want to turn off monitoring during work?” Over a period of usage, embodiments may self-optimize based on feedback and may search for moments at favorite locations to capture sound recordings and avoid sensitive locations.

It will therefore be appreciated that determination of a subject being in a target psychological state may be based on subject location. For instance, responsive to detecting a user is at a favorite location, it may be automatically inferred that the subject is in a happy mental state (wherein a happy mental state matches the target psychological state).

Heart rate (and/or heart rate variability), galvanic skin response, muscle tension (EMR), or detecting certain brainwaves (e.g. alpha waves) via head worn electrodes are other parameters that may be monitored to detect the occurrence of a target psychological state (e.g. relaxed mental/physical state). For example, if the subject's heart rate is within a predetermined target range (e.g. 50-70 beats per minute), it may be automatically inferred that the subject is in a relaxed state (wherein a relaxed state matches the target psychological state). Further, in combination with the favorite location data, heart rate variability information may help identify “feel good” moments, e.g. by detecting peaks and drops of the heart rate. For instance, an algorithm may look for moments where the heart rate raises and drops down in combination with other parameters (e.g. weather, location, time, connections (e.g. nearby persons, etc.)).

Another potential parameter that may be monitored to detect the occurrence of a target psychological state (e.g. relaxed mental/physical state) is the establishment of short-range communication links with other device. Based on such links, it may be determined that the subject is meeting a friend or family member, for example. That is, embodiments may be configured to detect instances when the subject is close to a known 3^{rd} party device, and then infer the subject is happy. For instance, when the subject's system/device connects to another device via a short range communication link (e.g. Bluetooth link), an embodiment may recognize the connected device as belonging to a family member of the subject and infer that the subject is in a happy mood (wherein a happy mood matches the target psychological state). Further, this may be combined together with location and heart rate information.

Purely by way of example:

1. The system detects a paired device within a predetermined range (e.g. 2-3 m);

2. The system detects a favorite location (such as a cafe in town, a holiday destination, saved favorite location, etc.);

3. The system detects significant peaks then drops in the subject's heart rate.

4. The system records 15 seconds of sounds from the surrounding environment.

Through feedback-based (i.e. supervised) machine learning and sound recognition, the detection of a target psychological state by embodiments may be further refined.

Weather

Weather data associated with the subject's location can be leveraged to add pre-made sound samples to the final track. For example, a “System Sound Library” may contain numerous premade sound samples. For each weather condition, one or more representative sounds may be employed. Such as, when the subject's location was windy and rainy, an embodiment may sound samples that represent wind and rain in a literal and/or artistic manner. Such additional sounds may provide the benefit of helping to keep the final audio signal different each day.

One of the other main reason to look at the weather data is also to determine if the subject is experiencing a positive state. Amongst all the other data points (location, heart rate etc. . . . ) weather data adds another layer to the algorithm for detecting the “feel good” moments. (e.g. If the subject is at the park and it is sunny, that can be a “feel good” moment.) The algorithm looks at weather to both personalize the listening experience as well as to help the algorithm to decide the psychological state.

Time

Embodiments may enable a user/subject to determine and/or control the monitoring time period. Such control of automated monitoring and sound recording functionality may allow privacy to the user/subject. Exemplary control aspects may allow the pre-setting of times to turn off the monitoring function, and/or when a subject is notified about a recording, the subject may be able to decline/snooze the recording with a simple input gesture.

Subject's Sound Capturing

As an alternative and/or addition to automated sound capturing, embodiments may be configured to enable the user/subject to actively take a role in the audio recording. This may be facilitated via a inputs and/or gestures, such as pressing a button to record 10-15 seconds (or longer) of audio. Every time the user/subject manually records audio, feedback-based machine learning algorithms maybe employed to learn from the monitored parameter values at the time, such as location, heart rate, weather, etc.

User's Sample Sounds

Some embodiments may be adapted to cater for a subject saving favorite sound samples into a library/database of sound samples. Such a “User Sample Sound” library may then contains short sound samples from the media files that are encountered by the user throughout the day. These are media files that the user likes and save to the “User Sample Sound” library, such as voice messages, videos, and music content that are encountered throughout the day via mobile chat apps, music, and video streaming services.

Sound Library

After audio recordings are captured by the subject's active participation and/or the automated system, they can be saved in a “Sound Library”. The content of this library may then contain layers of sounds created by the user and gathered by the environmental and physiological data. Such sounds may, for example, be categorized into three main categories: System Sample Sounds, Audio Recordings, Sample Sound Library.

— System Sample Sound Library

System sample sounds are pre-made sleep-inducing samples, including pink noise, binaural beats, ocean sounds and/or ambient music for example.

— Audio Recordings

The Audio Recordings library contains the sounds that are captured automatically throughout the day (e.g. responsive to determining the occurrence of a target psychological state). When the psychological state of the subject is detected as matching a target psychological state, a short (e.g. 5-20 seconds) audio recording of the surrounding environment is captured and saved into the audio recordings library. Embodiments may also enable a subject to actively capture audio recordings, and these may also be stored in the same library. Favorite/preferred audio recordings can also be saved to the Sample Sound library to customize other listening experiences on demand.

— Sample Sound Library

The Sample Sound library may be actively created by a user/subject. Similar to the Audio Recordings library, content wise it may have environmental sounds, conversations captured with the people around, and music sounds that are both captured from an environment as well added by the user to “Sample Sound Library”. The difference here is that these sounds are not solely active sound recordings of users' environment, but additional sound resources. Any suitable media content may be added to the user sample sound library on-demand. For instance, a user/subject may receive a video from a friend and see funny video content on social media during the day. The sound content of these media files may be added to the Sample Sound Library on demand.

Sound Mixing

Embodiments may be configured to mix together sleep inducing sounds and audio recordings to deliver a sleep-inducing sound mix (i.e. audio signal) comprising sound content gathered during the preceding monitoring time period (e.g. preceding daytime). The System Sample Sounds library, which comprises sleep-inducing sounds, may be used to create the foundation (i.e. base track) of the sleep-inducing sound mix. The presence of the sleep inducing sounds may be configured to be more dominant then the audio recordings to ensure the sound experience will induce sleep, and none of the audio recordings intrude or negatively impact the sleep-inducing nature of the sound mix. In some embodiments, the sleep inducing sounds may be longer in duration than the rest of the mixed sounds/recordings (e.g. to give room to a subject to slow down thinking and relax after hearing personal memories). Also, the System Sample Sound may be shaped by various parameters.

When curating (e.g. mixing) the audio signal, the daily average heart rate variability may set the tempo of the sleep-inducing sound mix in direct proportion between the range of 50-60 beats per minute, or a person's identified or measured resting heart rate. This rhythmic element may be created by binaural beats, and both the tempo and the sound frequencies may be configured to help to reduce the heart rate and relax breathing for stress reduction.

Also, the subject's location and the daily major weather forecasts may shape the listening experience characteristics by adding a related sound them. For instance, if the weather is windy, and the user passes through a city park during the day, the generation of the audio signal may comprise adding “City Park” sample and wind sounds to the final mix.

Breathing Exercise(s) may also be incorporated into the audio signal. For instance, a paced breathing exercise may be implement using sound cues added to the final mix. Depending on a selected breathing exercise, breathing guidance may be provided by marking every first beat of the inhale and exhale moments in concordance with a selected theme. e.g. Resonant breathing exercise: 6 seconds inhale-6 seconds exhale—A subtle bird sound on concrete theme singing every 6 seconds.

The user/subject may also configure the balance of each sound component in some embodiments. Such user/subject-driven may adjusts each main element in terms of duration and/or intervals. For example, a user/subject may choose the dominance of the components via a simple graphical user interface.

From the above-described embodiments and examples, it will be understood that the proposed concepts may be particularly relevant to medical facilities (e.g. hospitals) and/or care facilities (e.g. assisted living homes) where many users may be involved in a subject care process. In particular, embodiments may be of yet further relevance to academic hospitals.

It is to be understood that the above examples and embodiments are only illustrative and the ordering of one or more of the steps may be modified in alternative embodiments.

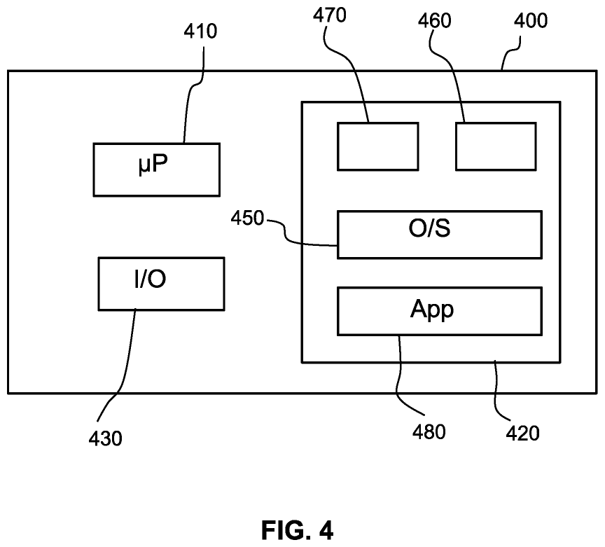

By way of further example, FIG. 4 illustrates an example of a computer 400 within which one or more parts of an embodiment may be employed. Various operations discussed above may utilize the capabilities of the computer 400. For example, one or more parts of a system for generating a sleep-inducing audio signal for a subject may be incorporated in any element, module, application, and/or component discussed herein. In this regard, it is to be understood that system functional blocks can run on a single computer or may be distributed over several computers and locations connected across a cloud-based system (e.g. connected via internet). That is, at least part of the system and/or sound recordings may be stored and executed in one or more cloud-based systems, of which the computer 400 may be part.

The computer 400 includes, but is not limited to, PCs, workstations, laptops, PDAs, palm devices, servers, storages, and the like. Generally, in terms of hardware architecture, the computer 400 may include one or more processors 410, memory 420, and one or more I/O devices 430 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 410 is a hardware device for executing software that can be stored in the memory 420. The processor 410 can be virtually any custom made or commercially available processor, a central processing unit (CPU), a digital signal processor (DSP), or an auxiliary processor among several processors associated with the computer 400, and the processor 410 may be a semiconductor based microprocessor (in the form of a microchip) or a microprocessor.

The memory 420 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 420 may incorporate electronic, magnetic, optical, and/or other types of storage media. Note that the memory 420 can have a distributed architecture, where various components are situated remote from one another, but can be accessed by the processor 410.

The software in the memory 420 may include one or more separate programs, each of which comprises an ordered listing of executable instructions for implementing logical functions. The software in the memory 420 includes a suitable operating system (O/S) 450, compiler 460, source code 470, and one or more applications 480 in accordance with exemplary embodiments. As illustrated, the application 480 comprises numerous functional components for implementing the features and operations of the exemplary embodiments. The application 480 of the computer 400 may represent various applications, computational units, logic, functional units, processes, operations, virtual entities, and/or modules in accordance with exemplary embodiments, but the application 480 is not meant to be a limitation.

The operating system 450 controls the execution of other computer programs, and provides scheduling, input-output control, file and data management, memory management, and communication control and related services. It is contemplated by the inventors that the application 480 for implementing exemplary embodiments may be applicable on all commercially available operating systems.

Application 480 may be a source program, executable program (object code), script, or any other entity comprising a set of instructions to be performed. When a source program, then the program is usually translated via a compiler (such as the compiler 460), assembler, interpreter, or the like, which may or may not be included within the memory 420, so as to operate properly in connection with the O/S 450. Furthermore, the application 480 can be written as an object oriented programming language, which has classes of data and methods, or a procedure programming language, which has routines, subroutines, and/or functions, for example but not limited to, C, C++, C #, Pascal, BASIC, API calls, HTML, XHTML, XML, ASP scripts, JavaScript, FORTRAN, COBOL, Perl, Java, ADA, .NET, and the like.

The I/O devices 430 may include input devices such as, for example but not limited to, a mouse, keyboard, scanner, microphone, camera, etc. Furthermore, the I/O devices 430 may also include output devices, for example but not limited to a printer, display, etc. Finally, the I/O devices 430 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 430 also include components for communicating over various networks, such as the Internet or intranet.

If the computer 400 is a PC, workstation, intelligent device or the like, the software in the memory 420 may further include a basic input output system (BIOS) (omitted for simplicity). The BIOS is a set of essential software routines that initialize and test hardware at startup, start the O/S 450, and support the transfer of data among the hardware devices. The BIOS is stored in some type of read-only-memory, such as ROM, PROM, EPROM, EEPROM or the like, so that the BIOS can be executed when the computer 400 is activated.

When the computer 400 is in operation, the processor 410 is configured to execute software stored within the memory 420, to communicate data to and from the memory 420, and to generally control operations of the computer 470 pursuant to the software. The application 480 and the O/S 450 are read, in whole or in part, by the processor 410, perhaps buffered within the processor 410, and then executed.

When the application 480 is implemented in software it should be noted that the application 480 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 480 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

A single processor or other unit may fulfill the functions of several items recited in the claims.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor. Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A method for generating a sleep-inducing audio signal for a subject, the method comprising:
monitoring a psychological state of a subject during a predetermined time period to detect if the psychological state of the subject matches a target psychological state;
responsive to detecting that the psychological state of the subject matches a target psychological state, obtaining an audio recording of the surrounding environment of the monitored subject, wherein the audio recording is captured at a detection time during which the psychological state of the subject matches the target psychological state; and
generating an audio signal for the subject based on the audio recording and one or more predetermined sleep inducing sounds.

2. The method of claim 1, further comprising:
responsive to detecting that the psychological state of the subject matches a target psychological state, determining a weather condition experienced by the subject at the
detection time and identifying a weather sound based on the determined weather condition, and wherein generating an audio signal for the subject is further based on the weather sound.

3. The method of claim 1, wherein monitoring a psychological state of the subject comprises monitoring at least one of:
subject location;
one or more vital signs of the subject;
one or more short-range communication links established with a computing device carried by the subject;
subject brainwaves;
environmental data;
inputs signals from the subject;
physiological parameters of the subject;
scheduled activities for the subject;
physical activity of the subject;
human-computer interaction patterns of the subject;
facial expressions from the subject; and
weather conditions experienced by the subject.

4. The method of claim 1, wherein generating (170) an audio signal for the subject is further based on predetermined sound cues for indicating respiration actions.

5. The method of claim 1, wherein generating (170) an audio signal comprises mixing the audio recording and the one or more predetermined sleep inducing sounds.

6. The method of claim 5 wherein mixing comprises defining a tempo of the audio signal based on at least one of: a preference of the subject; and detected value of a physiological parameter of the subject.

7. The method of claim 1, wherein monitoring (110) is controlled responsive to an indication or instruction provided by the subject.

8. The method of claim 1, wherein obtaining (130) an audio recording of the surrounding environment of the monitored subject comprises: capturing sound with a microphone carried by the subject.

9. The method of claim 1, wherein the one or more predetermined sleep inducing sounds comprise at least one of:
pink noise;
binaural beats;
ocean sounds;
ambient music; and
nature sounds.

10. The method of claim 1, further comprising communicating (180) the generated audio signal to a sleep inducing apparatus.

11. A computer program product comprising computer program code means which, when executed on a computing device having a processing system, cause the processing system to perform all of the steps of the method according to claim 1.

12. A system for generating a sleep-inducing audio signal for a subject, the system comprising:
a monitoring component configured to monitor a psychological state of a subject during a predetermined time period to detect if the psychological state of the subject matches a target psychological state;
an interface configured, responsive to detecting that the psychological state of the subject matches a target psychological state, to obtain an audio recording of the surrounding environment of the monitored subject, wherein the audio recording is captured at a detection time during which the psychological state of the subject matches the target psychological state;
an audio processor configured to generate an audio signal for the subject based on the audio recording and the one or more predetermined sleep inducing sounds.

13. The system of claim 12, wherein the audio processor comprises an audio mixer configured to mix the audio recording and the one or more predetermined sleep inducing sound.

14. The system of claim 12, further comprising:
an output interface configured to communicating the generated audio signal to a sleep inducing apparatus.

15. A sleep inducing apparatus comprising a system for generating a sleep-inducing audio signal for a subject according to claim 12.
